(19) United States
(12) Patent Application Publication
(10) Pub. No.: US 2016/0067286 A1
(43) Pub. Date: Mar. 10, 2016

(54) Formulation for Accelerating Wound Healing, Preparation Method and Administering Method of The Same

(71) Applicant: Ting-Chen Hung, Kaohsiung City (TW)

(72) Inventors: Ting-Chen Hung, Kaohsiung City (TW); Chuan-Ching Lan, Kaohsiung City (TW); Chia-Chi Lu, Kaohsiung City (TW); Cheng-Ruei Liu, Kaohsiung City (TW)

(21) Appl. No.: 14/510,579

(22) Filed: Oct. 9, 2014

(30) Foreign Application Priority Data
Sep. 10, 2014 (TW) 103131096

(51) Int. Cl. A61K 35/44; C12N 5/071

(52) U.S. Cl. CPC A61K 35/44; C12N 5/0692; C12N 2502/28; C12N 2506/11; C12N 2501/998; C12N 2501/65

(57) ABSTRACT

Disclosed is a formulation for accelerating wound healing, preparation method and administering method of the same. A formulation for accelerating wound healing comprises cell culture medium which is obtained by culturing transfected endothelial progenitor cells which are acquired by transfecting microRNA let-7g into endothelial progenitor cells. And the preparation method of formulation for accelerating wound healing comprises the isolating step, the transfecting step and the culturing step. A method for accelerating wound healing is implemented by administering a therapeutically effective amount of the formulation to an organism's wound.

### FIELD OF THE INVENTION

The present invention relates to a formulation, and more particularly to a formulation for accelerating wound healing, preparation method and administering method of the same.

### BACKGROUND OF THE INVENTION

Diabetes mellitus (commonly referred to as diabetes) is a common chronic disease in the world in which the body is in malfunction to convert glucose into energy because the body can not produce enough insulin or can not use insulin effectively in a process of glucose conversion. Diabetes is prone to cause diverse complications, including vascular lesion. High blood sugar causes hardening of the red blood cell membrane, making it difficult for blood to pass through capillaries into the wound tissue. Moreover, the hemoglobin is less likely to release the oxygen in a diabetic's body, resulting in tissue hypoxia and malnutrition. Besides, high blood sugar can also affect fibroblast and epithelial cell proliferation. A diabetic wound consequently tends to stall in the inflammatory phase, thereby developing into a chronic ulcer.

Currently, treatments for diabetic wound healing mainly include hyperbaric oxygen therapy, activated carbon dressing with silver and platelet-derived growth factor gel. Hyperbaric oxygen therapy is a treatment during which a patient is placed in a hyperbaric chamber that is pressurized. The pressure inside the chamber is maintained at 2.5 atmospheres absolute pressure (ATA). The patient enclosed within the chamber breathes pure oxygen through an oxygen mask so as to raise the oxygen concentration in blood, which then improves tissue hypoxia, promotes wound healing and enhances the ability of white blood cells to sterilize. However, this approach may cause damage to the lung, the eardrum and paranasal sinuses due to high pressure. Besides, spasm may be a symptom found as a result from the high concentrations of oxygen, and vision changes may be caused with the lens becoming swollen after the treatment. Furthermore, the treatment approach is extremely inconvenient for the patient since it cannot be self-administered in the home. Activated carbon dressing with silver is composed of PET nonwoven fabric, silver-coated activated carbon fiber cloth and polyethylene membrane. When the dressing touches wound exudate, it is found blood, exudate and bacteria can be absorbed by virtue of favorable absorption properties of the activated carbon fiber itself. Simultaneously, silver ion is capable of destroying cell membrane and nucleus of bacteria to achieve sterilization. In addition, far-infrared of activated carbon fiber is effectual in promoting blood circulation and accelerating metabolism, thus shortening wound healing time. However, the product entails a long period of time for diabetic wound healing. Moreover, coating the silver nano particles uniformly on the activated carbon fiber cloth is a complex and difficult manufacturing process, and the cost of silver nano particle is high, so consequently it results in increases in the product costs. Business competitiveness is thereby reduced. Moreover, there is another scheme that uses platelet-derived growth factor gel containing recombinate human platelet-derived growth factors for accelerating wound healing by virtue of its growth factor contained therein. However, the costs are expensive.

### SUMMARY OF THE INVENTION

Thus, an object of the present invention is to provide a formulation for accelerating wound healing, preparation method and administering method of the same in which the formulation can be prepared by virtue of a simple and relatively low-cost preparation method and can be applied in the patient anywhere and anytime.

The primary object of the present invention is to provide a formulation for accelerating wound healing, comprising: cell culture medium which is obtained by culturing transfected endothelial progenitor cells which are acquired by transfecting microRNA let-7g into endothelial progenitor cells (EPCs).

According to an embodiment of the present invention, the conditioned medium is endothelial basal medium (EBM) prior to the culture of transfected endothelial progenitor cells.

According to an embodiment of the present invention, the formulation further comprises at least one pharmaceutically acceptable carrier, diluent or excipient.

According to an embodiment of the present invention, the excipient is glycerol.

Another object of the invention is to provide a preparation method of formulation for accelerating wound healing, comprising steps of: isolating mononuclear cells from human whole blood and then seeding the mononuclear cells onto human-fibronectin-coated plates to obtain endothelial progenitor cells; transfecting microRNA let-7g into the endothelial progenitor cells to acquire transfected endothelial progenitor cells; and culturing the transfected endothelial progenitor cells for a predetermined time to obtain the formulation for accelerating wound healing.

According to an embodiment of the present invention, the culturing step comprises: seeding the transfected endothelial progenitor cells into endothelial growth medium-2 (EGM-2) for 7 days; on the 7^{th} day, replacing the EGM-2 with EBM and continuing culturing for 2 days; and collecting the EBM to obtain the formulation for accelerating wound healing.

According to an embodiment of the present invention, in the culturing step, the predetermined time is 6 to 10 days.

According to an embodiment of the present invention, the isolating step comprises using Histopaque-1077 to isolate the mononuclear cells from human whole blood by a method of density gradient centrifugation.

Another object of the invention is to provide a method for accelerating wound healing, comprising: administering a therapeutically effective amount of the formulation as claimed in claim 1 or claim 3 to an organism's wound.

According to an embodiment of the present invention, the organism is a diabetic patient.

By way of the technical means adopted by the present invention, in the present invention, the endothelial progenitor cells and microRNA let-7g are specifically chosen to prepare the formulation for accelerating wound healing together because the endothelial progenitor cells can differentiate into vascular endothelial cells to repair the incomplete vascular endothelium and accelerate angiogenesis and the microRNA let-7g is capable of protecting endothelial cells, curing and preventing arteriosclerosis and so on in the past research.

Thus, let-7g is transfected into the endothelial progenitor cells thereby regulating their expression. And the transfected endothelial progenitor cells are cultured in the culture medium so as to enable the factor with the ability for accelerating wound healing to be released from the endothelial progenitor cells. The culture medium not only is less expensive to the patient but also is provided with good effect in wound healing, so it can be applied widely in the patient whose wound is not prone to heal such as the diabetic patient. Moreover, the formulation of the present invention is used by administering the formulation directly to the patient's wound so it is convenient to be used anywhere and anytime.

### BRIEF DESCRIPTION OF THE DRAWINGS

FIG. 1 is a flow chart illustrating a formulation for accelerating wound healing according to an embodiment of the present invention;

FIG. 2 is a schematic diagram illustrating a diabetic mice wound model in an experiment for testing effect of the formulation used in healing wound of the diabetic mice according to the embodiment of the present invention;

FIG. 3 is an actual photograph illustrating the healing degree of the diabetic mice wound in each group in the experiment for testing effect of the formulation used in healing wound of the diabetic mice according to the embodiment of the present invention.

FIG. 4 is a line chart illustrating a trend of healing rate of the diabetic mice wound in each group in the experiment for testing effect of the formulation used in healing wound of the diabetic mice according to the embodiment of the present invention.

### DETAILED DESCRIPTION OF THE PREFERRED EMBODIMENTS

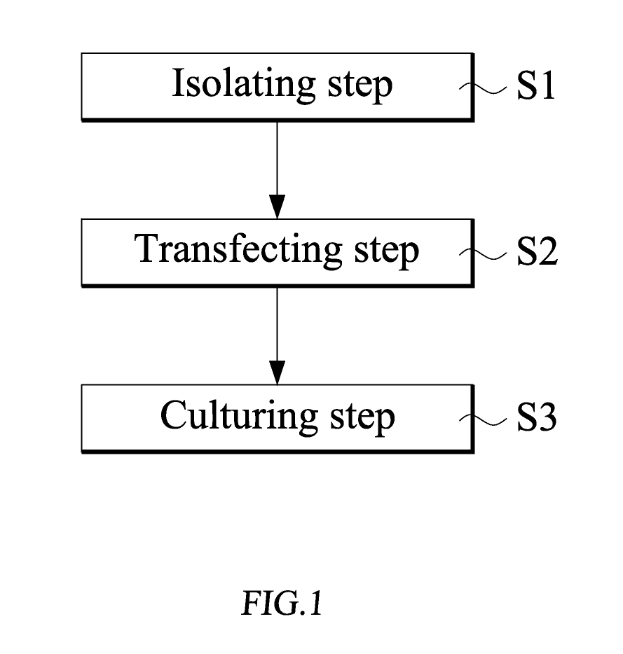

Embodiments are described in detail below with reference to the FIG. 1, and the description is used for explaining the embodiments of the present invention only, but not intended to limit the described embodiments of the present invention.

According to an embodiment of the present invention, a formulation for accelerating wound healing includes cell culture medium which is obtained by culturing transfected endothelial progenitor cells which are acquired by transfecting microRNA let-7g into endothelial progenitor cells (EPCs).

Specifically, the conditioned medium which is endothelial basal medium (EBM) prior to the culture of transfected endothelial progenitor cells is used for culturing endothelial progenitor cells specifically.

The formulation of the present invention further includes at least one pharmaceutically acceptable carrier, diluent or excipient for good stability, for a purpose of being used widely and reducing its irritation. For example, the formulation is preferable to turn from liquid state to semi-liquid state by adding glycerol with its viscosity property for the convenience of external use.

In this embodiment, a preparation method of formulation for accelerating wound healing includes steps of:

(1) isolating mononuclear cells from human whole blood and then seeding the mononuclear cells onto human-fibronectin-coated plates to obtain endothelial progenitor cells (S1);
(2) transfecting microRNA let-7g into the endothelial progenitor cells to acquire transfected endothelial progenitor cells (S2); and
(3) culturing the transfected endothelial progenitor cells for a predetermined time to obtain the formulation for accelerating wound healing (S3).

Specifically, in this embodiment, the endothelial progenitor cells are obtained by using gradient reagent Histopaque-1077 to isolate the mononuclear cells from human whole blood by a method of density gradient centrifugation. Thereafter, 10 ml gradient reagent Histopaque-1077 and 10 ml human whole blood are added to a 50 cc centrifuge tube to mix together and then the 50 cc centrifuge tube is centrifuged under 700g for 30 minutes. After being centrifuged, the buffy-coat layer which is mainly composed of mononuclear cells is collected. Then, the buffy-coat layer cells are suspended within 30 ml DMEM solution and are centrifuged under 250g for 10 minutes. After being centrifuged, the upper clean layer is removed while the cell pellets are retained. Then, the cell pellets are suspended within DMEM solution and centrifuged, in which the procedure is repeated for 2 to 3 times to obtain the mononuclear cell pellets. Lastly, the mononuclear cell pellets are resuspended with endothelial growth medium-2 (EGM-2) and seeded at a density of 3×10⁶ cells/ml onto a human-fibronectin-coated plate to obtain the endothelial progenitor cells.

In the transfecting step, 75 ng let-7g is diluted in the 100 μl culture medium and mixed well by vortexing. Then, 3 μl transfection reagent is added to the diluted let-7g, mixed well and incubated for 5 to 10 minutes at temperature between 15-25° C. to allow formation of transfection complexes. After that, the transfection complexes are added to the endothelial progenitor cells, and after 3 hours' incubation the culture medium is added to complete the transfection.

In the culturing step, the let-7g transfected endothelial progenitor cells are seeded into endothelial growth medium-2 for 7 days, and on the 7^{th} day the endothelial growth medium-2 is replaced with the endothelial basal medium and culturing is continued for 2 days. Finally, the endothelial basal medium is collected to obtain the formulation for accelerating wound healing. However, the present invention is not limited to this. The culturing period of the transfected endothelial progenitor cells can be 6 to 10 days according to the cell condition, the transfection efficiency or other experimental demands.

A method for accelerating wound healing by using the formulation for accelerating wound healing in the present invention is implemented by administering a therapeutically effective amount of the formulation or the formulation with acceptable carrier, diluent or excipient to an organism's wound, wherein the organism is a diabetic patient.

The following is an experiment of the formulation for accelerating wound healing used in the treatment of diabetic mice. The main procedures of the experiment include the diabetic animal model establishment, the diabetic animal wound model establishment, treatment of diabetic animal wound and statistical analysis.

First, diabetes is induced in C57BL/6J mice with streptozotocin (STZ) which is capable of destroying islet β cells. In this embodiment, the streptozotocin is injected into intraperitoneal of 8 to 12-week mice with dosage of 50 mg/kg body weight for 7 consecutive days and sugar level in these mice is controlled around the level of 150-200 mg/dL. After 20 mice are induced diabetes, of which six mice are classified as EBM group, seven mice are classified as let-7g(−)-EPC group and 7 mice are classified as let-7g (+)-EPC group for the subsequent experiment. Specifically, in the EBM group, the diabetic mice wound are treated with endothelial basal medium; in the let-7g(−)-EPC group, the diabetic mice wound are treated with cultured endothelial progenitor cells endothelial basal medium; and in the let-7g(+)-EPC group, the diabetic mice wound are treated with cultured let-7g transfected endothelial progenitor cells endothelial basal medium.

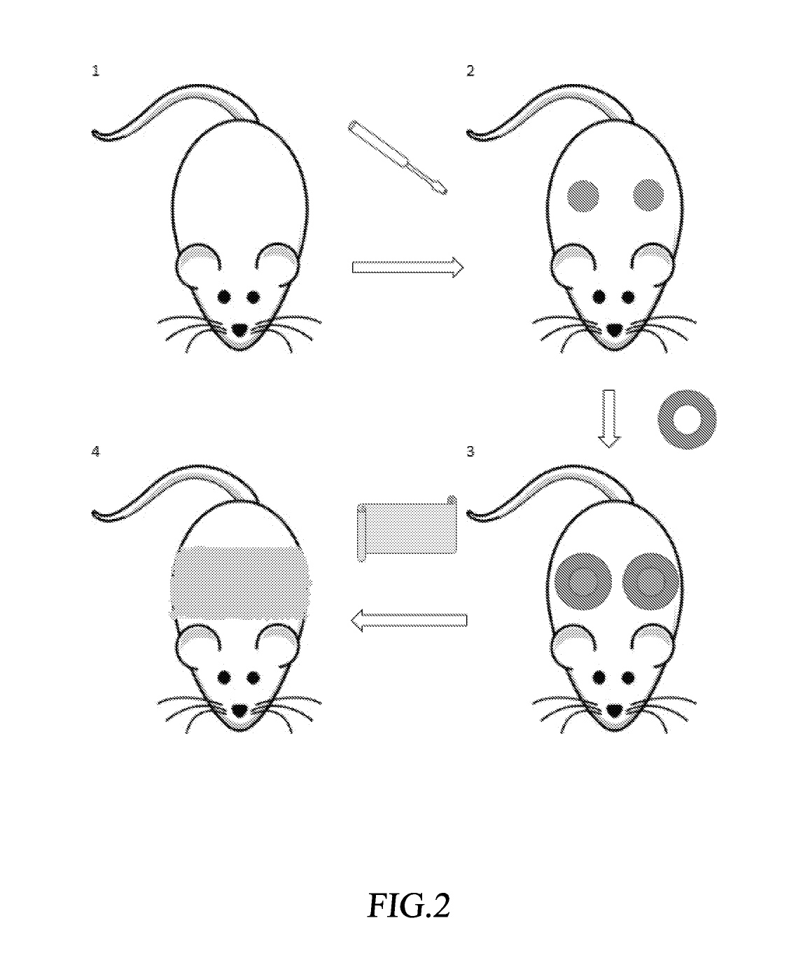

Referring to FIG. 2, after establishing the diabetic mice model, the dorsal surface for the wound formation of the diabetic mice is shaved to remove all the hair and disinfected. And two bilateral and deep panniculus carnosus wounds are formed on the dorsum of the diabetic mice with a 6-mm biopsy tool. Then, donut-shaped silicon splints are placed and fixed on the wound periphery and an occlusive dressing is covered on the wounds.

After establishing the diabetic mice wound, 50 μl endothelial basal medium, medium of endothelial progenitor cells and medium of let-7g transfected endothelial progenitor cells are respectively mixed with glycerol with 1:1 ratio and are separately used to treat the right wound of the mice in the EBM group, the let-7g(−)-EPC-CM group and the let-7g(+)-EPC-CM group as taking as a experimental group while leave the left wound of the mice without any treatment as taking as a control group, wherein the control group is used as comparing the wound healing rate with the experimental group. The frequency of treatment is everyday and the experiment is stopped until the wound is healed completely or due to other factors.

There has been significant difference between the three groups in the experiment by use the formulation for accelerating wound healing of the present invention with respect to the diabetic mice wound, so the present experiment is stopped on the 9^{th} day earlier than expected.

The photographs of wound area are taken every day with digital imaging equipment in the experiment. And pixel area is analyzed by using image-J analytic system to obtain wound remnant ratio, wound healing rate and wound healing rate difference. The wound remnant ratio is defined as a ratio of daily wound area divided by initial wound area. And the wound healing rate is defined as a rate that is obtained by minimizing the wound remnant ratio from 100%. Wound healing rate difference is defined as absolute subtraction value of the wound healing rate between the control group and the experimental group.

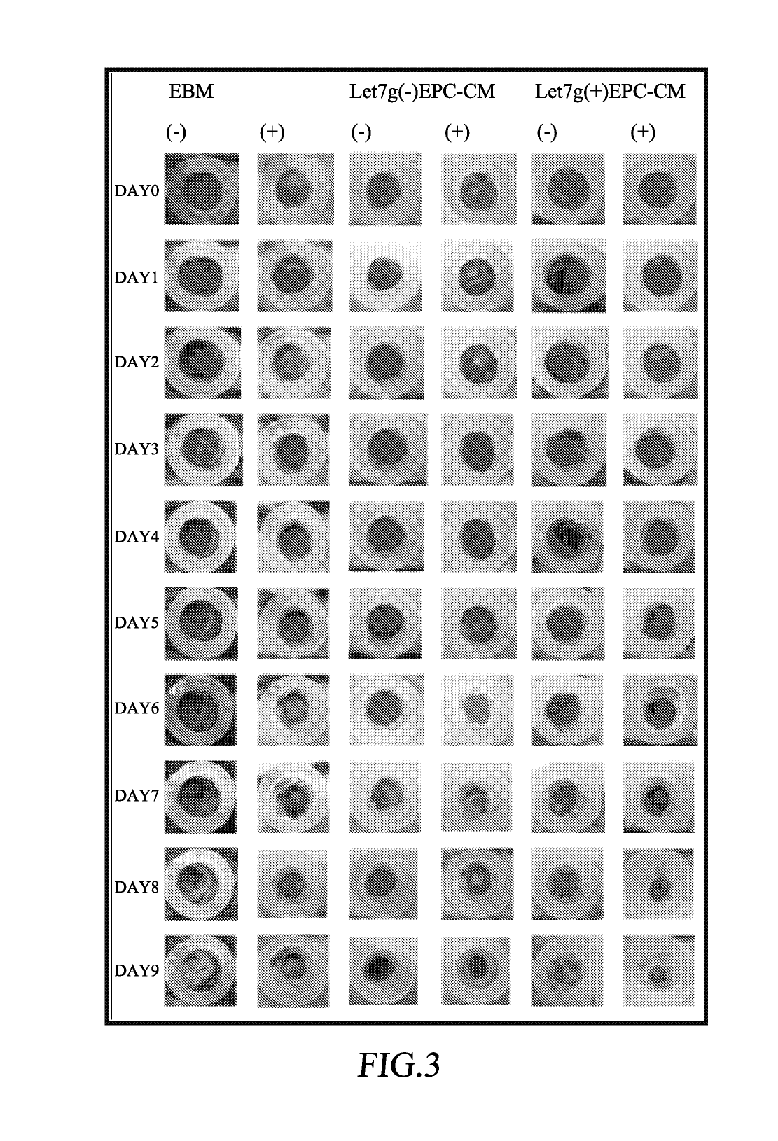

As shown in FIG. 3, actual pictures of wound healing condition of the mice in the each group reveals the experimental group in the let-7g(+)-EPC-CM group possessing the best wound healing effect.

As shown in table 1, the separate analysis of control groups and experimental groups in the three different groups on the wound healing rate with McNemar analysis reveals all the control groups possessing the better effect on the wound healing rate as compared to the experimental groups.

TABLE 1
McNenar test analysis of wound healing within each group †

| | Wound healing rate (%)(mean ± SD) | | | | | | | | |
|---|---|---|---|---|---|---|---|---|---|
| | EBM group (n = 6) | | | Let-7g(−)EPC-CM group (n = 7) | | | Let7g(+)EPC-CM group (n = 7) | | |
| | (−) | (+) | P value | (−) | (+) | P value | (−) | (+) | p-value |
| Day 0 | 0 ± 0 | 0 ± 0 | — | 0 ± 0 | 0 ± 0 | — | 0 ± 0 | 0 ± 0 | — |
| Day 1 | 1.23 ± 0.8 | 3.40 ± 1.1 | 0.007 | 2.46 ± 0.9 | 5.04 ± 1.8 | 0.004 | 2.18 ± 0.9 | 7.14 ± 1.8 | 0.000 |
| Day 2 | 3.96 ± 1.1 | 6.87 ± 1.6 | 0.005 | 6.98 ± 1.5 | 10.17 ± 3.3 | 0.092 | 7.76 ± 0.9 | 12.95 ± 3.0 | 0.009 |
| Day 3 | 8.44 ± 1.4 | 14.70 ± 3.6 | 0.023 | 15.8 ± 1.6 | 19.03 ± 2.1 | 0.005 | 16.3 ± 1.5 | 25.16 ± 2.6 | 0.001 |
| Day 4 | 16.57 ± 3.4 | 23.34 ± 2.4 | 0.002 | 23.19 ± 2.7 | 26.27 ± 3.5 | 0.008 | 23.05 ± 2.4 | 40.41 ± 3.9 | 0.000 |
| Day 5 | 25.60 ± 3.3 | 29.39 ± 2.9 | 0.011 | 33.94 ± 2.0 | 43.51 ± 3.2 | 0.001 | 34.68 ± 3.6 | 56.66 ± 2.4 | 0.000 |
| Day 6 | 35.62 ± 2.4 | 41.12 ± 3.0 | 0.000 | 41.12 ± 1.9 | 54.56 ± 3.1 | 0.000 | 43.04 ± 3.3 | 64.11 ± 1.6 | 0.000 |
| Day 7 | 47.32 ± 1.6 | 53.57 ± 2.6 | 0.001 | 48.73 ± 1.8 | 65.91 ± 2.8 | 0.000 | 54.34 ± 1.9 | 76.20 ± 2.6 | 0.000 |
| Day 8 | 55.45 ± 2.0 | 63.27 ± 2.2 | 0.000 | 57.84 ± 2.4 | 74.69 ± 3.2 | 0.000 | 61.92 ± 1.8 | 85.56 ± 0.9 | 0.000 |
| Day 9 | 65.47 ± 1.6 | 75.49 ± 2.9 | 0.001 | 67.93 ± 2.2 | 82.15 ± 1.6 | 0.000 | 71.64 ± 2.6 | 93.35 ± 0.5 | 0.000 |

† SPSS analysis,
P < 0.05 is significant

As shown in table 2, the comparison of the wound healing rate between control groups and experimental groups with regard to the EBM group, the let-7g(−)-EPC-CM group and the let-7g(+)-EPC-CM group by use of Kruskal-Wall analysis has also revealed significant difference. Although the wound in the control group is healed spontaneously, since part of the culture medium applied in the experimental group is absorbed by the body, it is found the wound healing in the distal region such as the wound in the control group is also effectual through peripheral circulation.

TABLE 2
Kruskal-Wallis analysis of wound healing between group †

| | Wound healing or growth rate (%)(mean ± SD) | | | | | | | |
|---|---|---|---|---|---|---|---|---|
| | Control group (−) ^{a} | | | | Study group (+) ^{a} | | | |
| | EBM | EPC-CM ^{b} | Let7g-CM ^{c} | P value | EBM | EPC-CM | Let7g-CM | P value |
| Day 0 | 0 ± 0 | 0 ± 0 | 0 ± 0 | — | 0 ± 0 | 0 ± 0 | 0 ± 0 | — |
| Day 1 | 1.23 ± 0.8 | 2.46 ± 0.9 | 2.18 ± 0.9 | 0.062 | 3.40 ± 1.1 | 5.04 ± 1.8 | 7.14 ± 1.8 | 0.001 |
| Day 2 | 3.96 ± 1.1 | 6.98 ± 1.5 | 7.76 ± 0.9 | 0.000 | 6.87 ± 1.6 | 10.17 ± 3.3 | 12.95 ± 3.0 | 0.004 |
| Day 3 | 8.44 ± 1.4 | 15.8 ± 1.6 | 16.3 ± 1.5 | 0.000 | 14.70 ± 3.6 | 19.03 ± 2.1 | 25.16 ± 2.6 | 0.000 |
| Day 4 | 16.57 ± 3.4 | 23.19 ± 2.7 | 23.05 ± 2.4 | 0.000 | 23.34 ± 2.4 | 26.27 ± 3.5 | 40.41 ± 3.9 | 0.000 |
| Day 5 | 25.60 ± 3.3 | 33.94 ± 2.0 | 34.68 ± 3.6 | 0.000 | 29.39 ± 2.9 | 43.51 ± 3.2 | 56.66 ± 2.4 | 0.000 |
| Day 6 | 35.62 ± 2.4 | 41.12 ± 1.9 | 43.04 ± 3.3 | 0.000 | 41.12 ± 3.0 | 54.56 ± 3.1 | 64.11 ± 1.6 | 0.000 |
| Day 7 | 47.32 ± 1.6 | 48.73 ± 1.8 | 76.17 ± 1.8 | 0.000 | 53.57 ± 2.6 | 65.91 ± 2.8 | 76.20 ± 2.6 | 0.000 |
| Day 8 | 55.45 ± 2.0 | 57.84 ± 2.4 | 61.92 ± 1.8 | 0.000 | 63.27 ± 2.2 | 74.69 ± 3.2 | 85.56 ± 0.9 | 0.000 |
| Day 9 | 65.47 ± 1.6 | 67.93 ± 2.2 | 71.64 ± 2.6 | 0.000 | 75.49 ± 2.9 | 82.15 ± 1.6 | 93.35 ± 0.5 | 0.000 |

† SPSS software,
P < 0.05 is significant
^{a} EBM group n = 6, let7g(−)EPC-CM group n = 7, let7g(+)EPC-CM group n = 7
^{b} EPC-CM means let-7g(−)EPC conditioned medium
^{c} let-7g-CM means let-7g(+)EPC conditioned medium

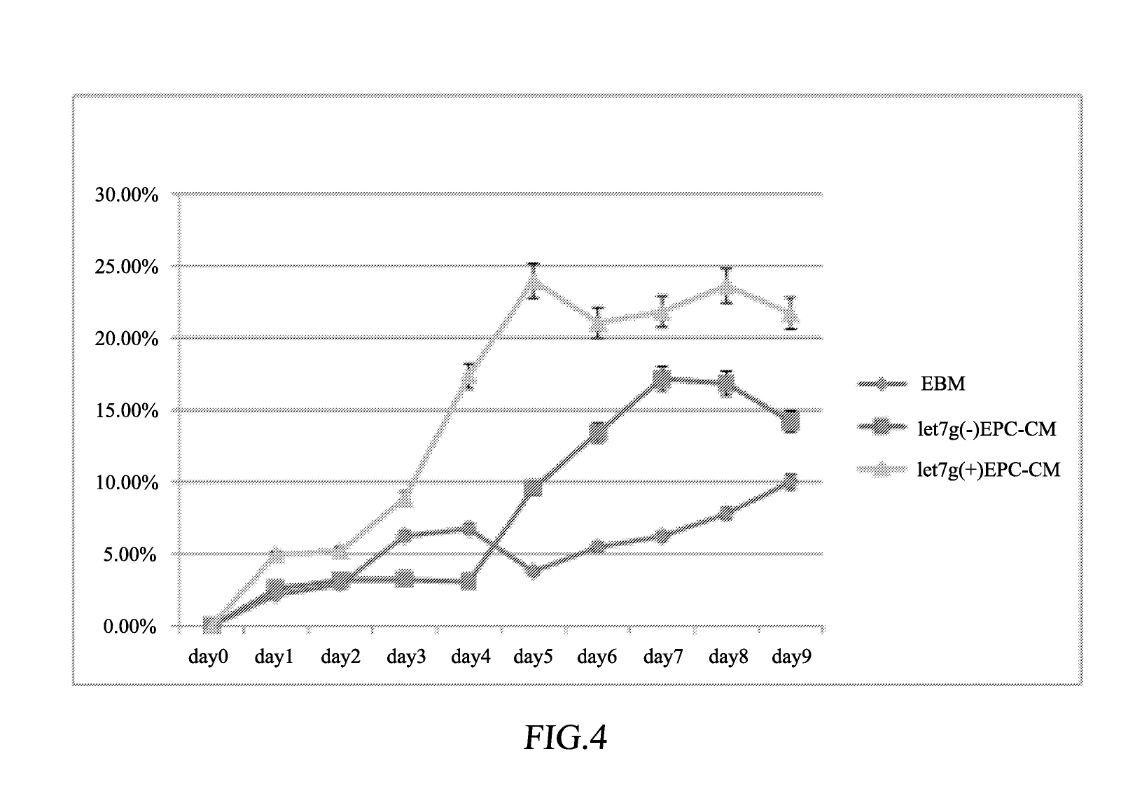

In order to further compare the therapeutic effects of the EBM group, the let-7g(−)-EPC-CM group and the let-7g(+)-EPC-CM group, the following table 3 to 6 are analysis by comparing the wound healing rate difference among the all three groups and comparing the wound healing rate difference one by one by use of the Kruskal-Wallis analysis and Mann-Whitney (M-W U) analysis. Referring to FIG. 4, it can be found that the let-7g(+)-EPC-CM group has the best effect on diabetic wound healing and the EBM group is the worst according to the trends of the wound healing rate difference of EBM group, the let-7g(−)-EPC-CM group and the let-7g(+)-EPC-CM group. Thus, the cell culture medium by culturing the let-7g transfected endothelial progenitor cells possesses the best effect of accelerating wound healing.

TABLE 3
Kruskal-Wallis analysis of wound healing rate difference †

| | Healing rate difference (%)(mean ± SD) | | | |
|---|---|---|---|---|
| | | Let7g(−)- | Let7g(+)- | |
| | EBM | EPC-CM | EPC-CM | |
| | (n = 6) | (n = 7) | (n = 7) | p-value |
| Day 0 | 0 ± 0 | 0 ± 0 | 0 ± 0 | — |
| Day 1 | 2.17 ± 1.2 | 2.58 ± 1.5 | 4.96 ± 1.5 | 0.029 |
| Day 2 | 2.91 ± 1.5 | 3.19 ± 4.2 | 5.20 ± 3.5 | 0.779 |
| Day 3 | 6.25 ± 4.7 | 7.23 ± 2.0 | 8.86 ± 3.5 | 0.039 |
| Day 4 | 6.76 ± 1.6 | 8.08 ± 2.1 | 17.36 ± 3.6 | 0.011 |
| Day 5 | 3.78 ± 2.4 | 9.58 ± 3.9 | 23.97 ± 4.9 | 0.003 |
| Day 6 | 5.49 ± 2.9 | 13.42 ± 2.9 | 21.07 ± 4.6 | 0.001 |
| Day 7 | 6.24 ± 2.3 | 17.17 ± 1.3 | 21.83 ± 1.8 | 0.001 |
| Day 8 | 7.82 ± 2.0 | 16.84 ± 3.5 | 23.64 ± 1.9 | 0.001 |
| Day 9 | 10.01 ± 3.1 | 14.21 ± 3.3 | 21.71 ± 2.1 | 0.005 |

† SPSS software,
p < 0.05 is significant

TABLE 4
M-W U analysis of wound healing rate difference
by EBM vs let7g(−)EPC-CM †

| | Wound healing rate difference (%)(mean ± SD) | | |
|---|---|---|---|
| | EBM | Let7g(−)EPC-CM | |
| | (n = 6) | (n = 7) | p-value ^{a} |
| Day 0 | 0 ± 0 | 0 ± 0 | — |
| Day 1 | 2.17 ± 1.2 | 2.58 ± 1.5 | 0.937 |
| Day 2 | 2.91 ± 1.5 | 3.19 ± 4.2 | 0.937 |
| Day 3 | 6.25 ± 4.7 | 7.23 ± 2.0 | 0.180 |
| Day 4 | 6.76 ± 1.6 | 8.08 ± 2.1 | 0.041 |
| Day 5 | 3.78 ± 2.4 | 9.58 ± 3.9 | 0.004 |
| Day 6 | 5.49 ± 2.9 | 13.42 ± 2.9 | 0.004 |
| Day 7 | 6.24 ± 2.3 | 17.17 ± 1.3 | 0.002 |
| Day 8 | 7.82 ± 2.0 | 16.84 ± 3.5 | 0.002 |
| Day 9 | 10.01 ± 3.1 | 14.21 ± 3.3 | 0.065 |

† SPSS software,
p < 0.05 is significant

TABLE 5
M-W U analysis of difference by EBM vs. let7g(+)EPC-CM †

| | Wound healing rate difference (%)(mean ± SD) | | |
|---|---|---|---|
| | EBM | Let7g(+)EPC-CM | |
| | (n = 6) | (n = 7) | p-value ^{a} |
| Day 0 | 0 ± 0 | 0 ± 0 | — |
| Day 1 | 2.17 ± 1.2 | 4.96 ± 1.5 | 0.005 |
| Day 2 | 2.91 ± 1.5 | 5.20 ± 3.5 | 0.534 |
| Day 3 | 6.25 ± 4.7 | 8.86 ± 3.5 | 0.731 |
| Day 4 | 6.76 ± 1.6 | 17.36 ± 3.6 | 0.051 |
| Day 5 | 3.78 ± 2.4 | 23.97 ± 4.9 | 0.008 |
| Day 6 | 5.49 ± 2.9 | 21.07 ± 4.6 | 0.001 |
| Day 7 | 6.24 ± 2.3 | 21.83 ± 1.8 | 0.001 |
| Day 8 | 7.82 ± 2.0 | 23.64 ± 1.9 | 0.001 |
| Day 9 | 10.01 ± 3.1 | 21.71 ± 2.1 | 0.005 |

† SPSS software,
p < 0.05 is significant

TABLE 6
M-W U analysis of difference by let7g(−)EPC-CM
vs let7g(+)EPC-CM

| | Wound healing rate difference (%)(mean ± SD) | | |
|---|---|---|---|
| | Let7g(−)EPC-CM | Let7g(+)EPC-CM | |
| | (n = 7) | (n = 7) | p-value ^{a} |
| Day 0 | 0 ± 0 | 0 ± 0 | — |
| Day 1 | 2.58 ± 1.5 | 4.96 ± 1.5 | 0.101 |
| Day 2 | 3.19 ± 4.2 | 5.20 ± 3.5 | 0.731 |
| Day 3 | 7.23 ± 2.0 | 8.86 ± 3.5 | 0.005 |
| Day 4 | 8.08 ± 2.1 | 17.36 ± 3.6 | 0.014 |
| Day 5 | 9.58 ± 3.9 | 23.97 ± 4.9 | 0.035 |
| Day 6 | 13.42 ± 2.9 | 21.07 ± 4.6 | 0.051 |
| Day 7 | 17.17 ± 1.3 | 21.83 ± 1.8 | 0.022 |
| Day 8 | 16.84 ± 3.5 | 23.64 ± 1.9 | 0.035 |
| Day 9 | 14.21 ± 3.3 | 21.71 ± 2.1 | 0.022 |

† SPSS software,
p < 0.05 is significant

The above description should be considered as only the discussion of the preferred embodiments of the present invention. However, a person with an ordinary skill in the art may make various modifications to the present invention and those modifications still fall within the spirit and scope defined by the appended claims.

What is claimed is:

1. A formulation for accelerating wound healing, comprising:
cell culture medium which is obtained by culturing transfected endothelial progenitor cells which are acquired by transfecting microRNA let-7g into endothelial progenitor cells (EPCs).

2. The formulation as claimed in claim 1, wherein the conditioned medium is endothelial basal medium (EBM) prior to the culture of transfected endothelial progenitor cells.

3. The formulation as claimed in claim 1, further comprising at least one pharmaceutically acceptable carrier, diluent or excipient.

4. The formulation as claimed in claim 3, wherein the excipient is glycerol.

5. A preparation method of formulation for accelerating wound healing, comprising steps of:
isolating mononuclear cells from human whole blood and then seeding the mononuclear cells onto human-fibronectin-coated plates to obtain endothelial progenitor cells;
transfecting microRNA let-7g into the endothelial progenitor cells to acquire transfected endothelial progenitor cells; and
culturing the transfected endothelial progenitor cells for a predetermined time to obtain the formulation for accelerating wound healing.

6. The preparation method as claimed in claim 5, wherein the culturing step comprises:
seeding the transfected endothelial progenitor cells into endothelial growth medium-2 (EGM-2) for 7 days;
on the 7th day, replacing the EGM-2 with EBM and continuing culturing for 2 days; and
collecting the EBM to obtain the formulation for accelerating wound healing.

7. The preparation method as claimed in claim 5, wherein in the culturing step, the predetermined time is 6 to 10 days.

8. The preparation method as claimed in claim 5, wherein in the isolating step, comprising using Histopaque-1077 to isolate the mononuclear cells from human whole blood by a method of density gradient centrifugation.

9. A method for accelerating wound healing, comprising: administering a therapeutically effective amount of the formulation as claimed in claim 1 to an organism's wound.

10. The method as claimed in claim 9, wherein the organism is a diabetic patient.
